(19) United States
(12) Patent Application Publication
(10) Pub. No.: US 2026/0174441 A1
(43) Pub. Date: Jun. 25, 2026

(54) DEVICES AND METHODS FOR OCCLUDING THE LEFT ATRIAL APPENDAGE

(71) Applicant: AtriCure, Inc., Mason, OH (US)

(72) Inventor: Frank M. FAGO, Mason, OH (US)

(73) Assignee: AtriCure, Inc., Mason, OH (US)

(21) Appl. No.: 19/536,613

(22) Filed: Feb. 11, 2026

Related U.S. Application Data
(63) Continuation of application No. PCT/US2024/042887, filed on Aug. 19, 2024.
(60) Provisional application No. 63/520,270, filed on Aug. 17, 2023.

(51) Int. Cl. A61B 17/122; A61B 17/072; A61B 17/29; A61B 34/00; A61B 34/30

(52) U.S. Cl. CPC A61B 17/1227; A61B 17/07207; A61B 17/29; A61B 34/30; A61B 34/71; A61B 2017/291; A61B 2017/2927; A61B 2017/2939

(57) ABSTRACT

Disclosed herein are systems and methods for applying an occlusion clip. The medical device can comprise a handle, an elongated shaft extending from the handle, the elongated shaft comprising a proximal end, a distal end, and a central lumen. The medical device can further comprise an end effector at the distal end of the elongated shaft, the end effector comprising a first jaw and a second jaw configured to engage with and deliver the occlusion clip to a target tissue, wherein the end effector comprises a link mechanism configured to move the first jaw and the second jaw relative to each other. The medical device can further comprise a plurality of cables extending through the central lumen. The medical device can further comprise an articulation plate coupled to the handle. The medical device can further comprise an articulation joint coupling the articulation plate and the elongated shaft.

### CROSS-REFERENCE TO RELATED APPLICATIONS

This application is a continuation of PCT Application No. PCT/US2024/042887, filed Aug. 19, 2024, which claims priority to U.S. Provisional Application No. 63/520,270, filed on Aug. 17, 2023, the entirety of which are incorporated by reference.

### FIELD OF TECHNOLOGY

The present disclosure relates generally to the field of left atrial appendage closure, and more specifically, closing the left atrial appendage with an occluding device.

### BACKGROUND

Current methods for left atrial appendage closure often comprise the use of scalpels, graspers, and needle drivers to amputate the appendage and sew up the remaining wound. Such devices can be more universal and can be used for multiple surgical procedures. However, such devices can necessitate more operator training and require a higher skill level to use.

In addition, current devices are often restricted by their diameter, restricting every aspect of the device. For example, some current devices can be introduced via a 12 mm trocar. This restriction can result in additional overall procedure time.

Therefore, there remains a need for methods and devices for occluding a left atrial appendage that are intuitive to use to minimize confusion. There also remains a need for devices that can fit through a small trocar.

### SUMMARY

A medical device for applying an occlusion clip is disclosed herein. The medical device can comprise a handle, an elongated shaft extending from the handle, the elongated shaft comprising a proximal end, a distal end, and a central lumen. The medical device can further comprise an end effector at the distal end of the elongated shaft, the end effector comprising a first jaw and a second jaw configured to engage with and deliver the occlusion clip to a target tissue, wherein the end effector comprises a link mechanism configured to move the first jaw and the second jaw relative to each other. The medical device can further comprise a plurality of cables extending through the central lumen. The medical device can further comprise an articulation plate coupled to the handle, wherein the articulation plate comprises a central opening and a plurality of peripheral openings, wherein each of the plurality of peripheral openings receives a screw, wherein each screw receives one of the plurality of cables for tensioning of the link mechanism. The medical device can further comprise an articulation joint coupling the articulation plate and the elongated shaft, the articulation joint configured to allow rotational movement between the handle and the elongated shaft.

The articulation joint can be a ball-and-socket joint. The device can further comprise an O-ring configured to engage with the ball-and-socket joint and the articulation plate to lock the ball-and-socket joint to prevent articulation of the end effector. The plurality of peripheral openings can comprise a D-shaped cross-section.

Each screw can comprise a through hole therethrough, wherein the through hole comprises a first through hole diameter and a second through hole diameter, wherein the first through hole diameter is smaller than the second through hole diameter. A first ball and a second ball can be coupled to each of the plurality of cables, wherein the first ball comprises a first ball diameter smaller than the first through hole diameter, wherein the second ball comprises a second ball diameter larger than the first through hole diameter, wherein pulling the cable distally pulls the first ball through the through hole and traps the second ball in order to tension the cable. The first ball can be coupled to a ball-and-socket joint within the end effector.

The cable can be crimped by the screw such that the cable is restricted from moving axially. The end effector can further comprise a ball-and-socket joint. The ball-and-socket joint can comprise a plurality of openings to which each of the plurality of cables couples to. A ball of the ball-and-socket joint can comprise a slot, wherein a socket of the ball-and-socket joint comprises a pin, wherein the slot and the pin are coupled to prevent circumferential rotation of the ball-and-socket joint.

The link mechanism can comprise a plurality of linkage bars coupled to the first jaw and the second jaw, wherein the plurality of linkage bars transfer forces from the plurality of cables to the first jaw and the second jaw. The plurality of cables can each comprise a plurality of wires.

In other variations, a system for occluding a left atrial appendage of a patient is disclosed herein. The system can comprise a handle, an elongated shaft extending from the handle, the elongated shaft comprising a proximal end, a distal end, and a central lumen extending along a longitudinal axis of the elongated shaft. The system can further comprise an end effector at the distal end of the elongated shaft, the end effector comprising a first jaw and a second jaw configured to engage with and deliver an occlusion clip to a target tissue, wherein the end effector comprises a link mechanism configured to move the first jaw and the second jaw relative to each other. The system can further comprise a plurality of cables extending through the central lumen. The system can further comprise an articulation plate coupled to the handle. The system can further comprise an articulation joint coupling the articulation plate and the shaft, the articulation joint configured to allow rotational movement between the handle and the elongated shaft. The handle can comprise a lever portion configured to rotate in a first plane perpendicular to the longitudinal axis, wherein the lever portion is configured to rotate in a second plane perpendicular to the first plane, wherein rotation of the lever portion in the first plane moves the end effector in a yaw direction and rotation of the lever portion in the second plane moves the end effector in a pitch direction.

The handle can further comprise a grip portion configured for gripping by an operator with one or more fingers, wherein the articulation plate is between the grip portion and the lever portion. The lever portion can rotate 45 degrees in a first direction and 45 degrees in a second direction in the first plane, wherein the lever portion rotates 45 degrees in a first direction and 45 degrees in a second direction in the second plane. The handle can further comprise a thumb portion extending from the lever portion, wherein the thumb portion extends at a 45-degree angle from the longitudinal axis of the elongated shaft.

In another variation, the medical device can comprise a handle, an elongated shaft extending from the handle, the elongated shaft comprising a proximal end, a distal end, and a central lumen. The medical device can further comprise an end effector at the distal end of the elongated shaft, the end effector comprising a first jaw and a second jaw configured to engage with and deliver the occlusion clip to a target tissue. The medical device can further comprise a plurality of cables extending through the central lumen. The medical device can further comprise an articulation plate coupled to the handle, wherein the articulation plate comprises a central opening and a plurality of peripheral openings, wherein each of the plurality of peripheral openings receives a screw. The medical device can further comprise an articulation joint coupling the articulation plate and the elongated shaft, the articulation joint configured to allow rotational movement between the handle and the elongated shaft. The medical device can further comprise an articulation lock system within the handle, wherein the articulation lock system comprises a ring configured to engage the articulation joint and the articulation plate such that the articulation joint and the articulation plate are locked, locking the end effector.

The articulation lock system can further comprise a brake actuator plate, wherein the brake actuator plate is configured to release the ring from engagement with the articulation joint and the articulation plate when the occlusion clip is released. An elastic member can be coupled to the brake actuator plate.

### BRIEF DESCRIPTION OF THE DRAWINGS

FIG. 1 illustrates a side view of a device for applying a clip to occlude the left atrial appendage.

FIG. 2A illustrates one variation of a tensioning screw used in conjunction with the articulation plate.

FIG. 2B illustrates one variation of a screw assembly and a corresponding method of tensioning the peripheral cables.

FIG. 2C illustrates another variation of a screw assembly and a corresponding method of tensioning the peripheral cables.

FIG. 2D illustrates another variation of a screw assembly and a corresponding method of tensioning the peripheral cables in which the screw further comprises a crimping element that surrounds the cable.

FIG. 3A illustrates one variation of a handle in accordance with the present invention.

FIG. 3B illustrates a front view of the articulation plate comprising a central opening and peripheral openings surrounding the central opening.

FIG. 3C illustrates a diagram of the thumb portion in relation to the longitudinal axis of the elongated shaft 106.

FIGS. 3D and 3E illustrate views of variations of the handle being held by a user.

FIG. 4 illustrates an articulation lock designed to secure the end effector as desired by the user.

FIGS. 5A and 5B illustrate the distal ball-and-socket joint that enables the end effector to accomplish pitch and yaw at one position.

FIG. 6 illustrates a handle according to another variation of the device.

FIGS. 7A-E illustrate the end effector at various positions during use.

FIG. 8 illustrates another variation of the device that provides rotation to the end effector.

### DETAILED DESCRIPTION

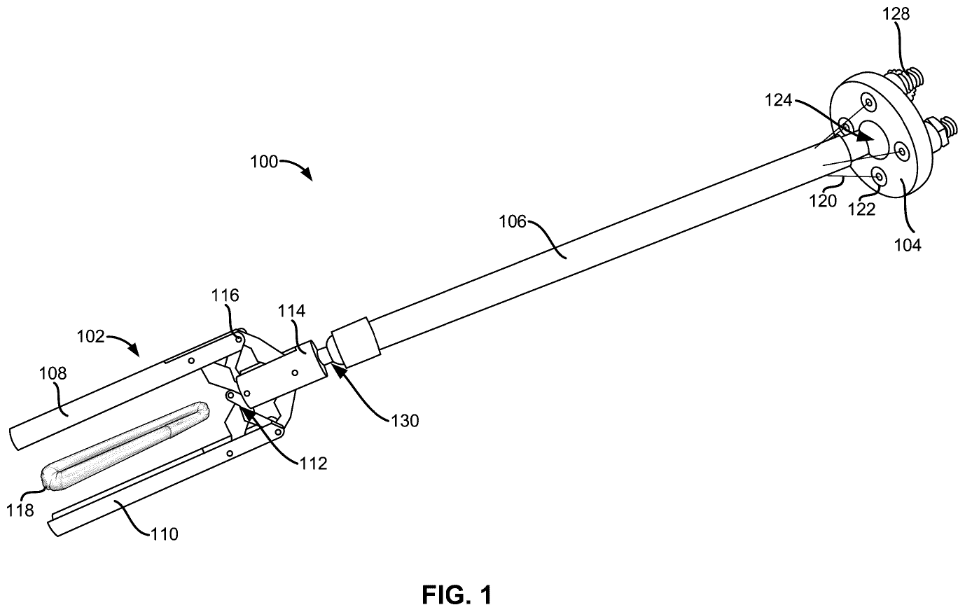

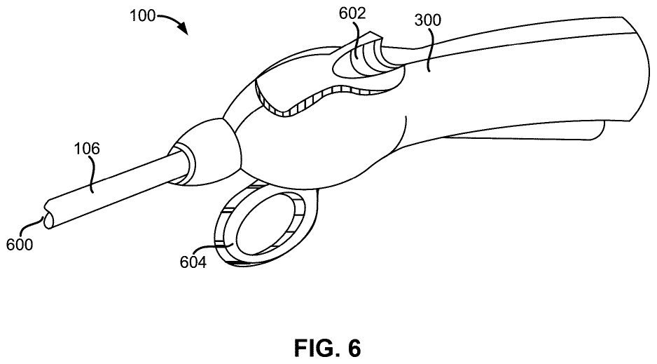

FIG. 1 illustrates a medical device 100 for applying an occlusion clip to occlude the left atrial appendage. The device 100 can comprise an end effector 102, an articulation plate 104 coupled to a handle (not shown), and an elongated shaft 106 comprising a longitudinal axis and a central lumen 600 (see FIG. 6) extending between the end effector 102 and articulation plate 104.

The end effector 102 can comprise a first jaw 108, a second jaw 110, and a linkage mechanism 112 that connects the jaws 108, 110 to a base portion 114. A plurality of pins 116 can be used to connect various parts of the linkage mechanism 112. The first jaw 108 and the second jaw 110 can extend from the linkage mechanism 112 and can move relative to each other to deliver a clip 118 around the left atrial appendage. Reference is made to commonly owned U.S. Pat. No. 9,861,371, the entirety of which is incorporated by reference.

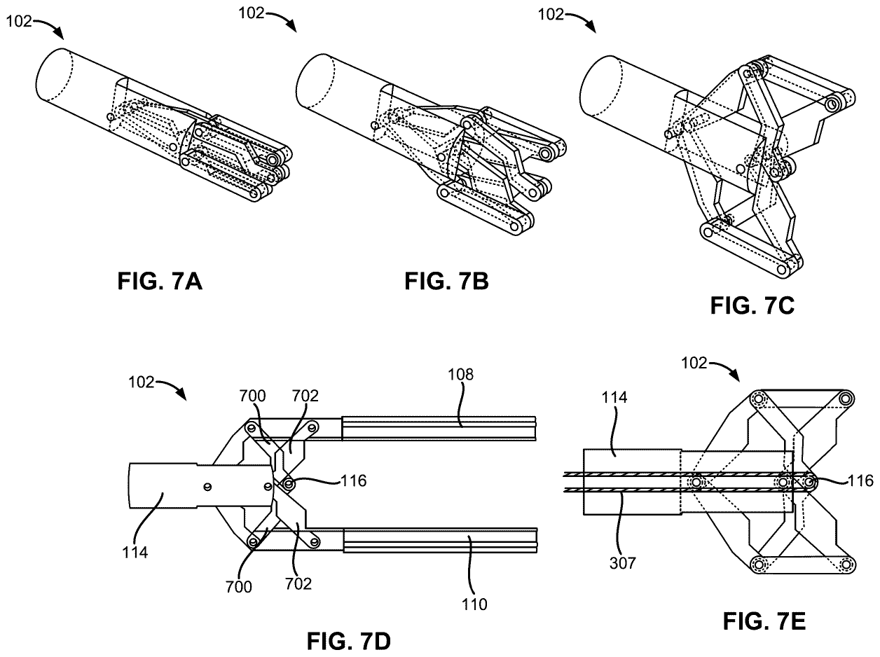

It should be understood that the end effector 102 can be used to apply clips at other target tissue or target areas of the anatomy, however. As will be described below, the linkage mechanism 112 can comprise a plurality of linkage bars configured to transition the jaws 108, 110 between a first, compact configuration and a second, expanded configuration (as seen in FIGS. 7A-E).

The end effector 102 can be configured to fit in a conventional port or trocar of about 5 mm to 7 mm (e.g., about 5.9 mm) commonly used in minimally invasive thoracic surgeries. A port or trocar size of this diameter is critical to reduce pain for the patient as the access between the ribs is tight and comprises nearby nerves. The elongated shaft 106 can thus comprise a diameter of about 5 mm to about 7 mm to fit through the trocar and to permit bending if necessary.

The end effector 102 in the first, compact configuration (see FIG. 7A) can have a diameter of about 5.84 mm. The end effector 102 can be configured to open clip sizes in the second, expanded configuration (see FIG. 7C) of about 20 mm in order to open the inside of the clip to approximately 14 mm.

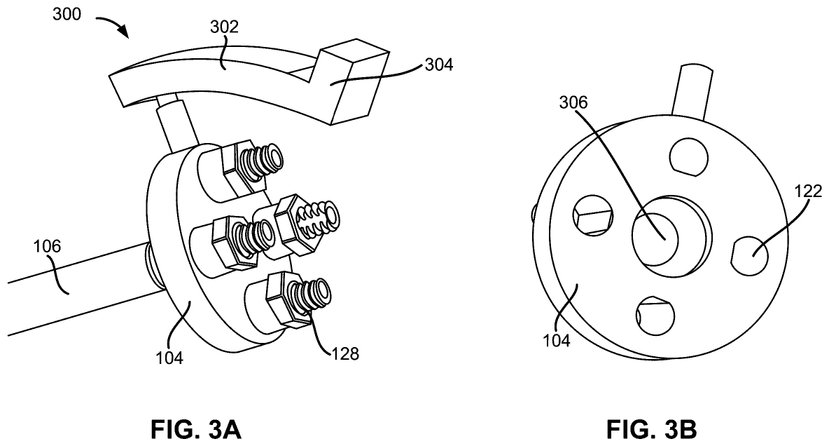

The elongated shaft 106 can comprise a plurality of clip opening cables 120 within the central lumen. The central cables 307 can extend through a central opening of the articulation plate 104 (see FIG. 3B) and into the handle such that the handle can control the first jaw 108 and the second jaw 110 moving with respect to each other, as will be described below.

A plurality of peripheral cables 120 can exit the elongated shaft 106 at the proximal end of the elongated shaft 106 and into one or more peripheral openings 122 of the articulation plate 104. The cables 120, 307 can thus extend from the linkage mechanism 112 to their respective components in the handle and can be used to control the end effector 102 via the handle. For example, the peripheral cables 120 can be used to articulate the end effector 102 in both a pitch and yaw direction with respect to a central plane of the device 100.

The articulation plate 104 and the elongated shaft 106 can comprise a proximal ball-and-socket joint 124 therebetween. A ball of the proximal ball-and-socket joint 124 can comprise an opening through the center to allow the opening and a plurality of cables to exit and interface with their control elements in the handle. The proximal ball-and-socket joint 124 can accordingly allow for rotation between the handle and the elongated shaft 106.

The plurality of peripheral cables 120 can exit the articulation plate 104 and to the handle through a plurality of screws 128 placed within the peripheral openings 122 of the articulation plate 104.

The end effector 102 and the elongated shaft 106 can comprise a distal ball-and-socket joint 130 therebetween. The distal ball-and-socket joint 130 can be articulated by the handle to rotate the end effector as desired. The distal ball-and-socket joint 130 can be locked by the handle when such movement is no longer desired. The distal ball-and-socket joint 130 can be as close to the end effector 102 as possible to reduce the dead space such the end effector 102 can provide articulation in the limited space between a trocar and the left atrial appendage.

The distal ball-and-socket joint 130 can be compact in order to operate in the limited space between the end of the trocar and the left atrial appendage. Accordingly, the distal ball-and-socket joint 130 can fit within a trocar of about 5 mm diameter. The stiffness of the distal ball-and-socket joint 130 can be optimized so the clip can be pushed to the base of the appendage during use.

Each of the cables 120, 307 (central cables or peripheral cables) can comprise a 1×7 wire (e.g., seven wires within one bundle) construction, however, other cable constructions can be used. The cables 120, 307 can comprise a diameter of about 0.016 inches. The cables 120, 307 can comprise a minimum breaking strength of about 30 lb. The cables 120, 307 can be made of stainless steel, though other materials can be used (e.g., aluminum, copper, titanium, tungsten, etc.).

In other variations, the cables 120, 307 can comprise various wire constructions which can vary in strength and flexibility. In some variations, the cables 120, 307 can comprise a 1×19 wire (e.g., nineteen wires within one bundle).

In other variations, the cables 120, 307 can comprise multiple cables made of multiple wires. For example, the cables 120, 307 can comprise a 3×7 configuration (21 total wires), a 7×7 configuration (49 total wires), a 7×19 configuration (133 total wires), or a 7×49 configuration (343 total wires).

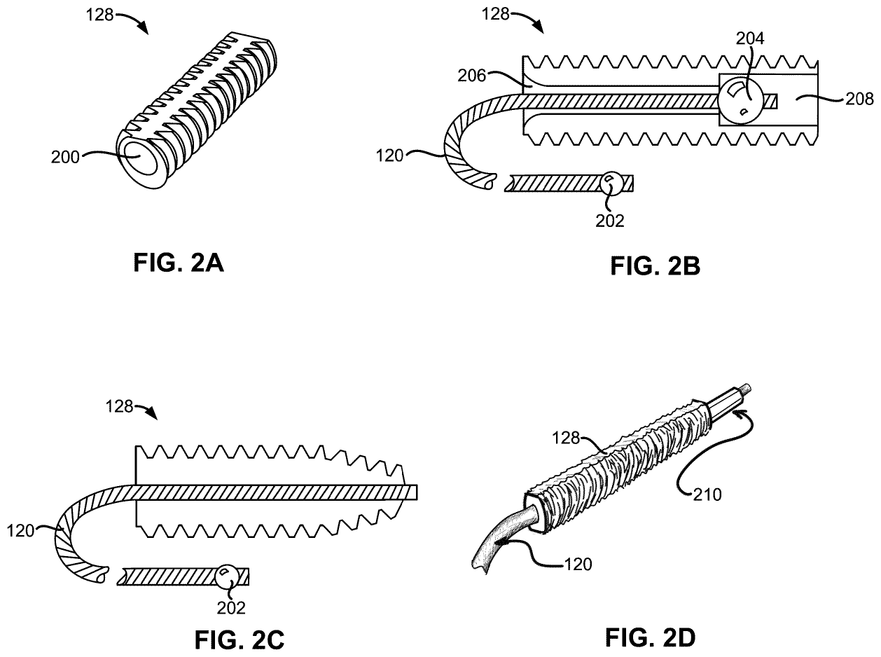

FIG. 2A illustrates one variation of the tensioning screw 128 used in conjunction with the articulation plate 104. The tensioning screw 128 can comprise an opening 200 therethrough. The opening 200 can accommodate the peripheral cables 120, controlling the tension of the peripheral cables 120 for articulation of the end effector 102.

One technical problem encountered by the applicants is that current devices are built from the bottom up, so access to the cables for tensioning is limited to the top side. A manufacturing fixture using a complex series of pulleys and weights can often be used to set the tension. The forces from the cable tension are resolved in the handles which are not completely fixed until a final ultrasonic welding step. This results in variations in tension, so the articulation forces are not consistent. One technical solution discovered by the applicants is to tension the peripheral cables 120 such that they are tight enough that the tension keeps end effector 102 rigid. At the same time, the peripheral cables should have some slack to decrease the friction within the device 100 such that the end effector 102 can still articulate. Accordingly, to ease manufacturing and to set the tension of the device 100 to desired levels, the screw assembly can be tensioned and aligned as a unit prior to being assembled into the handle. Such methods are described with respect to FIGS. 2B to 2D below.

FIG. 2B illustrates one variation of a screw assembly and a corresponding method of tensioning the peripheral cables 120. This variation comprises a screw with an opening 200 comprising two inner diameters. In this variation, the peripheral cables 120 can be prefabricated to approximately its functional length in order to eliminate a termination step during manufacturing. The peripheral cable 120 can comprise a distal ball 202 having a diameter of about 0.03 inches and a proximal ball 204 having a diameter of about 0.06 inches. The screw 128 can comprise an opening 200 having two inner diameters: a first through hole 206 and a second through hole 208, the second through hole 208 comprising a larger diameter than a diameter of the first through hole 206. The distal ball 202 can pass through the screw 128 while the proximal ball 204 is trapped by a counterbore (i.e., where the opening 200 changes diameter). The distal ball 202 then feeds through the elongated shaft 106 and is attached to the distal ball-and-socket joint 130. It should be understood that the plurality of peripheral cables 120 can comprise the same length since the articulation function of the device 100 occurs in the same plane.

The screw 128 can have an anti-rotation feature such as a flat to keep it from rotating as the cable is tensioned. The screw 128 can fit through a D-shaped cross-section peripheral opening 200 in the articulation plate 104 and the tension can be set with a nut on the opposite side of the articulation plate 104. When the screw 128 is aligned, the tension can be set by counting additional turns on the nuts. This way, various tensions can be quickly modified during development to determine the optimum user feel.

FIG. 2C illustrates another variation of a screw 128 assembly and a corresponding method of tensioning the peripheral cables in which the screw 128 comprises an opening 200 in which the peripheral cable 120 cannot move. During manufacturing, the screw 128 can be crimped to trap the cable in its place axially.

FIG. 2D illustrates another variation of a screw 128 assembly and a corresponding method of tensioning the peripheral cables in which the screw 128 further comprises a crimping element 210 that surrounds the peripheral cable 120. This sets the peripheral cable 120 in place when the correct tension is reached such that the peripheral cable 120 is unable to move axially.

One technical problem encountered by the applicants is maintaining end effector 102 rigidity while still providing articulation. One technical solution discovered by the applicants is to increase the pre-load on the peripheral cables 120. For example, if the articulation cable were not pre-loaded, a force on the end effector of 10 pounds of tension would stretch the cable about 0.05 inches, resulting in considerable end-effector backlash. However, if the cable had a starting tension of 10 pounds, the same load would not deflect the end effector at all. The friction between the proximal ball-and-socket joint 124 can also help the rigidity of the end-effector.

FIG. 3A illustrates one variation of a handle 300 in accordance with the present invention. The handle 300 can extend from the articulation plate 104 and can comprise a lever portion 302 and a thumb portion 304. The lever portion 302 can be configured to rotate in a first plane (e.g., a y-plane) perpendicular to the longitudinal axis of the elongated shaft 106 in order to move the end effector in a yaw direction. The lever portion 302 can be configured to rotate in a second plane perpendicular (e.g., a z-plane) to the first plane in order to move the end effector in a pitch direction.

FIG. 3B illustrates a front view of the articulation plate 104 comprising a central opening 306 and peripheral openings 122 surrounding the central opening 306. The central opening 306 can accommodate and mount a ball of the proximal ball-and-socket joint 124. The central cables 307 can enter and exit an opening of the ball to interface with their control elements in the handle 300 to opening and close the jaws 108, 110 of the end effector 102.

The peripheral cables 120 in the articulation plate 104 can be located further from the center than the peripheral cables 120 at the distal end near the end effector 102 (e.g., at the distal ball-and-socket joint 130). Accordingly, less angular movement in the articulation plate 104 can result in more movement at the end effector 102. For example, 10 degrees of articulation at the articulation plate 104 can be more than 20 degrees at the distal ball-and-socket joint 130.

The articulation plate 104 can comprise a round protrusion on an opposite side of the central opening 306 that can fit in between halves of the handle such that the articulation plate 104 is constrained. Accordingly, the articulation plate 104 can rotate laterally in order to provide the yaw direction control (right and left). The rotating moment force can be achieved by the lever portion 302 and the thumb portion 304 which is located about 1 inch from the proximal ball-and-socket joint. The end effector 102 can be moved in the pitch direction (up and down) when the lever portion 302 is moved axially respective to the elongated shaft 106. For example, pushing the lever forward can move the end-effector down with respect to the longitudinal axis. Likewise, the yaw controls can move the end effector 102 to the left when the thumb portion 304 is pushed to the left (with respect to the operator's point of view) to keep cable forces in line. In some variations, the articulation of the peripheral cables 120 can be reversed to change the articulation direction at the distal end of the device 100.

FIG. 3C illustrates a variation where the thumb portion is positioned at a 45-degree angle in front of the articulation plate 104. The thumb portion 304 can extend about 45 degrees from the longitudinal axis of the elongated shaft 106 such that it can share the motion for pitch and yaw of the end effector 102 at approximately the same force and distance for both (see e.g., FIG. 3C). This allows the user to control the end effector 102 in all directions from a single control point via their thumb (i.e., the user can use the thumb portion in a joystick-like manner).

FIGS. 3D and 3E illustrate a handle 300 comprising a control using a thumb portion 304 and a finger portion 308. The finger portion 308 can be used to add force to the control of the thumb portion 304, improving the ergonomics of the handle 300 for the user 310. The thumb portion 304 in FIG. 3D can be a bulb-shaped design. The thumb portion 304 in FIG. 3E can comprise a concentric ring design to facilitate omnidirectional functionality with the thumb portion 304 in a potentially wet environment. The handle 300 can further comprise a clip opening control 312 that can actuate the jaws 108, 110 moving in relation to each other.

For ergonomic purposes, the handle 300 can be designed such the articulation plate 104 is in line with the user's carpometacarpal joint in the thumb located near the wrist. The carpometacarpal joint is also a ball joint, and accordingly, keeping it in line with the proximal ball-and-socket joint 124 mechanism in the handle 300 can minimize unwanted relative motion between the user's thumb and the lever portion 302.

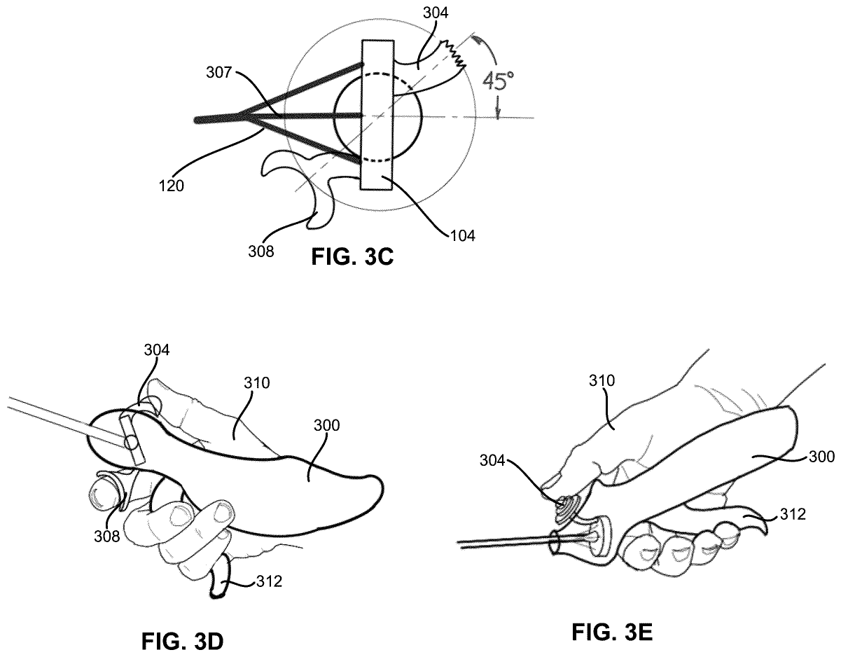

FIG. 4 illustrates an articulation lock system 400 (or brake system) designed to secure the end effector 102 as desired by the user. The proximal ball-and-socket joint 124 can be locked when movement of the end effector 102 is no longer desired. The articulation lock system 400 can be within the handle 300 and can employ a high-friction, elastomeric O-ring 402 that can be pressed against the proximal ball-and-socket joint 124 to prevent the end effector 102 from back-driving when force is applied to the clip at the distal end of the device 100.

As seen in FIG. 4, the O-ring 402 can be positioned around the ball 404 and within a wedge-shaped opening between the articulation plate 104 and the proximal ball 404, allowing a relatively small amount of force to push the O-ring 402 into the opening where the force is effectively multiplied. When the O-ring 126 engages the wedge-shaped opening between the articulation plate 104 and the proximal ball 404, the proximal ball-and-socket joint 124 can be locked from rotating in relation to the articulation plate 104. As the articulation plate 104 attempts to move, the friction generated tightens the O-ring 402 further, increasing the holding force. The end effector 102 can be locked when the O-ring 402 is in this position.

A brake actuator plate 406 can be connected to an elastic clip release spring 408 via a cable 410, such that when the clip is released, the lock is disengaged, enabling easy removal of the end effector 102 from the device 100.

The articulation lock system 400 is intended to increase friction sufficiently to stabilize the end effector 102. While in the locked position, the end effector 102 can still be repositioned by the user with the application of additional force. A cam-type mechanism (not shown) could retract the brake actuator plate 406, causing the O-ring 402 to disengage from the slot, allowing the lever portion 302 of the handle 300 to move with minimal force.

In some variations, to eliminate a control from the device, it can be possible to use friction and/or force applied by the operator to keep the end effector 102 in the desired position.

In some variations, the O-ring 402 can be linked to the spring 408 for clip deployment so when the clip 118 is deployed the O-ring 402 will disengage. This ensures that the end effector 102 is removable (i.e., not locked in place) such that the end effector 102 can be pulled straight during removal from the trocar. The O-ring 402 can engage rigid components such as the proximal ball-and-socket joint 124 in the handle 300.

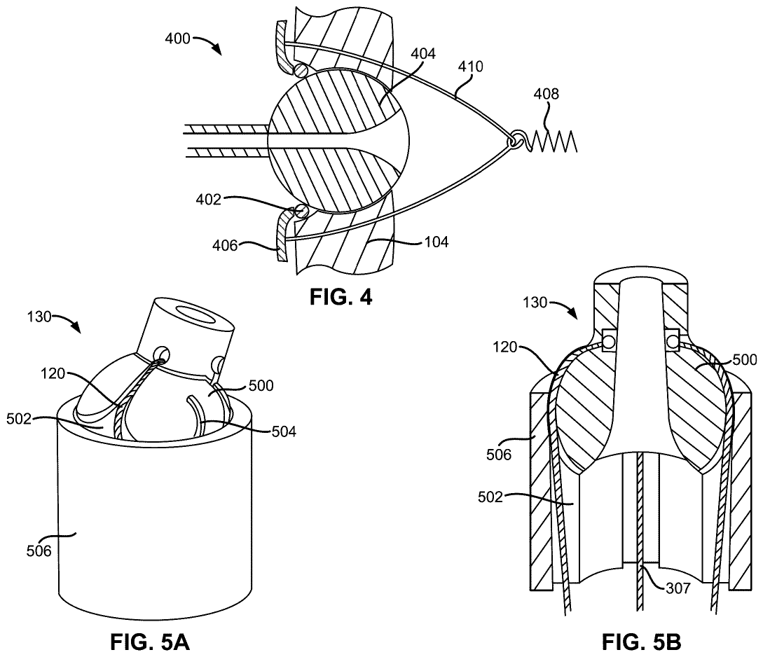

FIGS. 5A and 5B illustrate the distal ball-and-socket joint 130 that enables the end effector 102 to accomplish pitch and yaw at one position. This can reduce the overall dead space on the pivoting section of the end effector 102 by about 0.375 inches, which can be helpful as the distance between the appendage and the distal end of the trocar can be limited, especially in smaller patients.

The distal ball 500 can comprise cable slots 502 where the peripheral cables 120 couple to. The cable slots 502 can act as channels for the peripheral cables 120 that get wider at the equator and southern hemisphere proportionately, so articulation off center does not change the cable tension. In some variations, the length of the peripheral cables 120 does not change as the end effector 102 is pivoted to keep the articulation force constant.

A slot 504 on the ball and a pin at the equator of the socket (not shown) can prevent the distal ball 500 from rotating circumferentially. The peripheral cables 120 can always be in tension such that they will hold the distal ball-and-socket joint 130 is held together.

Since the peripheral cable 120 tension can be high, it is important to minimize friction between the distal ball 500 and the socket 506. Accordingly, the socket 506 can be made of materials such as nylon or polyetherimide to reduce friction and to provide adequate strength for the socket 506. Additionally, the surface finish and fit between the proximal ball 404 and socket 506 can impact the friction force to articulate.

In some variations, hypotubes can be crimped outside of the peripheral cables 120 to help maintain the end-effector rigidity, compensating for increased cable loads and subsequent cable stretch. In some variations, a tungsten alloy cable can be used to bolster the strength of the peripheral cables 120 by about 30% while increasing the flexibility of the cables.

FIG. 6 illustrates a handle 300 according to another variation of the device 100. The handle 300 can comprise an articulation lever 602 and a clip opening lever 604. The handle can be positioned at a proximal end of an elongated shaft. An end effector (not shown) can be positioned at a distal end of the elongated shaft 106.

The articulation lever 602 can be positioned at a top of the handle 300 such that the user can control the articulation lever 602 with their thumb while holding the handle 300 with one hand. The articulation lever 602 can be configured such that motion of the lever corresponds with the end effector so as to be intuitive to the user.

The clip opening lever 604 can comprise a ring shape on an underside of the handle 300 such that the user can control the clip opening lever 604 with one finger while holding the handle 300 with one hand. The clip opening lever 604 can provide control to open and close the clip without requiring substantial force from the user.

In some variations, the clip opening lever 604 can be a momentary control such that the end effector 102 only moves when the clip opening lever 604 is depressed. The opening 200 in the clip opening lever 604 can be used for the opening function, such that the other fingers of the user are available for the lock lever. The clip opening lever 604 can also be an on-off lock.

The clip opening lever 604 can transfer up to about 10 lbs of force over a distance of about 0.375 inches to the clip opening central cables 307. The stroke of the clip opening lever 604 can be a specified length such that adequate mechanical advantage can be achieved with the lever mechanism. The mechanical advantage can be a result of stroke length and force applied to the clip opening lever 604. The applied force on the clip opening lever 604 can be reduced proportionately to the travel distance.

The clip opening function can be achieved with only the index finger as the ergonomics of the handle 300 can allow as such. The index finger can hook the opening lever loop and provide a better grip on the device 100. In some variations, a 1-inch pull can provide a 3:1 reduction in force, and accordingly, the pulling force needed to open the clip can be about 3 to 4 pounds.

A clip release mechanism can provide tactile and auditable feedback to ensure the clip has positively been released. In some variations, the clip release mechanism can be a button on the clip opening lever 604.

A single control point can move the end effector 102 and allow for simultaneous motion. Since the articulation controls are simplified and can be done concurrently with the clip opening lever 604, in some variations, it may not be necessary to provide a separate lock for the articulation.

In some variations, the clip opening lever 604 can have a locking mechanism in the open position. To eliminate a separate button, the clip opening lever 604 can lock when the clip is open and can release if the clip opening lever 604 is pressed a second time.

FIGS. 7A to 7E illustrate the end effector 102 at various positions during use. The end effector 102 can comprise one or more proximal linkage bars 700 and one or more distal linkage bars 702 linked to each other and forming an opening to receive the clip 118 via the jaws 108, 110. The one or more linkage bars 700, 702 can pivot with respect to each other on one or more pins 116. The pins 116 can be about 0.03 inches in diameter and can be configured to minimize frictional losses. FIG. 7A illustrates a first, compact configuration and FIG. 7C illustrates a second, expanded configuration.

The two jaws 108, 110 can be positioned at an end of the linkage bars 700, 702. The clip attachment jaws 108, 110 can be substantially parallel with a longitudinal axis of the elongated shaft 106.

One or more linkage bars 700, 702 can actuate an opening link mechanism that can transfer perpendicular forces from a central cable 307 to the clip attachment jaws 108, 110, as seen in FIGS. 7D and 7E. The central cable 307 can pass through a lumen of the shaft and can couple to the one or more linkage bars 700, 702. The central cable 307 can comprise a diameter of about 0.016 inches and can loop over the opening linkage pin 116.

The end effector 102 can be configured to fit through a trocar of about 5 mm diameter and can take advantage of the fact that trocars are slightly larger than their nominal description.

The end effector 102 can open the clip to an aperture of about 14 mm. The clip and end effector 102 can articulate 30° from the central plane in both pitch (e.g., up and down) and yaw (e.g., left and right).

Each of the cables 307 can fit in a space of about 0.02 inches provided between a center of the linkage bars 700, 702. The cable 307 can be rigid such that the cable 307 remains on the pivot pin 116. The cable 307 can comprise a diameter of about 0.016 inches.

In some variations, the cables 307 can comprise a preformed “U” bend such that the cable 307 can straddle pivot pins 116 in the end effector 102. This configuration can double the cable strength to reduce stretching and to allow adequate safety factor to the end effector 102. The safety factor can be further increased using tungsten alloy wires.

Both free ends of the cable can be attached to a clip opening lever 604 in the handle 300. Since the preformed “U” shaped end of the opening cables can constrain the pivot pin 116, the end effector 102 can open symmetrically. In some variations, the cable 307 can also be mechanically attached to the pivot pin 116 or the proximal link bar. The load required to open the clip can be less than 10 pounds, and accordingly, a conventional cable can be used in some variations.

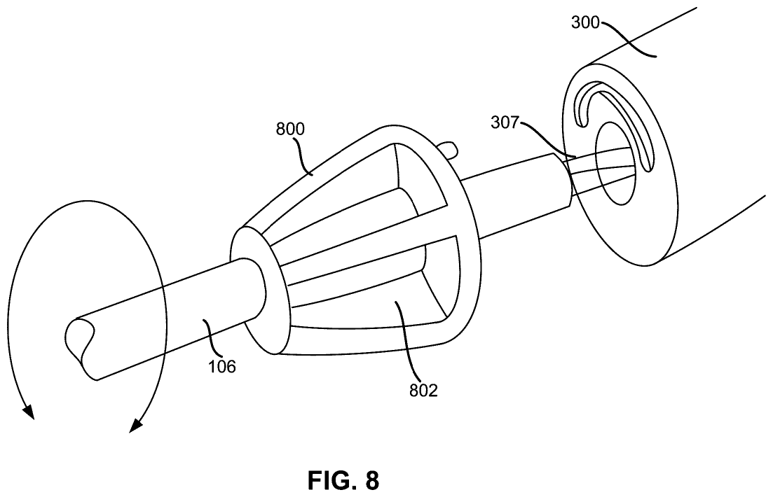

FIG. 8 illustrates another variation of the device 100 that provides rotation to the end effector 102. The device 100 can comprise a rotating cone 800 with one or more grooves 802 positioned thereon for improved ergonomics. The rotating cone 800 can be positioned on the elongated shaft 106 such that the elongated shaft 106 rotates when the user rotates the rotating cone 800. Accordingly, the end effector 102 is rotated as the central cables 307 within the elongated shaft 106 twist to accommodate the rotation. Rotation of the end effector 102 can be limited to about 180 degrees to prevent binding. The significant tension on the central cables 307 naturally provides resistance to prevent the elongated shaft 106 from rotating too freely. If a greater rotational locking force is desired, friction can be increased by using higher-friction materials or incorporating surface interlocking features to ensure more secure positioning.

The distal linkage bars 702 and the proximal linkage bars 700 can be in compression such that they are subjected to buckling loads. The linkage bars can be about 0.03 inches thick. A buckling resistance of the linkage bars 700, 702 can be increased by the thickness or the width of the linkage bars 700, 702. Further, the buckling resistance laterally can be improved by tight-fitting pinned joints. The proximal linkage bars 700 can be in tension and can be configured for resolving the loads and providing lateral stability. If needed, a proximal linkage bar 700 length can be slightly shorter than a distal linkage bar 702 length, causing the jaws of the end effector 102 to splay outwards to compensate for clearances or any deflection of the jaws 108, 110.

The linkage bars 700, 702 can be made from materials such as 17-4 stainless steel in order to have a safety factor to resolve the loads of opening the clip 118. In some variations, the end effector 102 can be made using a metal injection molding process or a metal 3-D printing process.

The end effector 102 can comprise two separate pivots, one for pitch and one for yaw, positioned about 0.375 inches apart one for pitch and one for yaw. The distance from the first pivot to the proximal side of the clip when the clip is closed can be about 1.5 inches. The end effector 102 can articulate in a 40-degree pitch and yaw in both directions from the center.

For a conventional left-sided, sixth intercostal rib space approach, the end effector typically requires minimal articulation from the centerline. However, variations in anatomy, right-side bilateral access and other future applications can necessitate some articulation. Since the elongated shaft 106 is small in diameter, it can be beneficial to allow the elongated shaft 106 to allow for slight bends.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations can be provided, or steps or operations can be eliminated or omitted from the described methods or processes to achieve the desired results. Moreover, any components or parts of any apparatus or systems described in this disclosure or depicted in the figures can be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings can be regarded in an illustrative rather than a restrictive sense.

Each of the individual variations or embodiments described and illustrated herein has discrete components and features which can be readily separated from or combined with the features of any of the other variations or embodiments. Modifications can be made to adapt a particular situation, material, composition of matter, process, process act(s) or step(s) to the objective(s), spirit, or scope of the present invention.

Methods recited herein can be carried out in any order of the recited events that is logically possible, as well as the recited order of events. Moreover, additional steps or operations can be provided or steps or operations can be eliminated to achieve the desired result.

Furthermore, where a range of values is provided, every intervening value between the upper and lower limit of that range and any other stated or intervening value in that stated range is encompassed within the invention. Also, any optional feature of the inventive variations described can be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter can conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

It will be understood by one of ordinary skill in the art that the various methods disclosed herein can be embodied in a non-transitory readable medium, machine-readable medium, and/or a machine accessible medium comprising instructions compatible, readable, and/or executable by a processor or server processor of a machine, device, or computing device. The structures and modules in the figures can be shown as distinct and communicating with only a few specific structures and not others. The structures can be merged with each other, can perform overlapping functions, and can communicate with other structures not shown to be connected in the figures. Accordingly, the specification and/or drawings can be regarded in an illustrative rather than a restrictive sense.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that can become obvious to those skilled in the art in view of this disclosure.

We claim:

1. A medical device for applying an occlusion clip, the medical device comprising:
a handle;
an elongated shaft extending from the handle, the elongated shaft comprising a proximal end, a distal end, and a central lumen;
an end effector at the distal end of the elongated shaft, the end effector comprising a first jaw and a second jaw configured to engage with and deliver the occlusion clip to a target tissue, wherein the end effector comprises a link mechanism configured to move the first jaw and the second jaw relative to each other;
a plurality of cables extending through the central lumen;
an articulation plate coupled to the handle, wherein the articulation plate comprises a central opening and a plurality of peripheral openings, wherein each of the plurality of peripheral openings receives a screw, wherein each screw receives one of the plurality of cables for tensioning of the link mechanism; and
an articulation joint coupling the articulation plate and the elongated shaft, the articulation joint configured to allow rotational movement between the handle and the elongated shaft.

2. The medical device of claim 1, wherein the articulation joint is a ball-and-socket joint.

3. The medical device of claim 2, further comprising an O-ring configured to engage with the ball-and-socket joint and the articulation plate to lock the ball-and-socket joint to prevent articulation of the end effector.

4. The medical device of claim 1, wherein the plurality of peripheral openings comprise a D-shaped cross-section.

5. The medical device of claim 1, wherein each screw comprises a through hole therethrough, wherein the through hole comprises a first through hole diameter and a second through hole diameter, wherein the first through hole diameter is smaller than the second through hole diameter.

6. The medical device of claim 5, further comprising a first ball and a second ball coupled to each of the plurality of cables, wherein the first ball comprises a first ball diameter smaller than the first through hole diameter, wherein the second ball comprises a second ball diameter larger than the first through hole diameter, wherein pulling the cable distally pulls the first ball through the through hole and traps the second ball in order to tension the cable.

7. The medical device of claim 6, wherein the first ball is coupled to a ball-and-socket joint within the end effector.

8. The medical device of claim 1, wherein the cable is crimped by the screw such that the cable is restricted from moving axially.

9. The medical device of claim 1, wherein the end effector further comprises a ball-and-socket joint.

10. The medical device of claim 9, wherein the ball-and-socket joint comprises a plurality of openings to which each of the plurality of cables couple.

11. The medical device of claim 9, wherein a ball of the ball-and-socket joint comprises a slot, wherein a socket of the ball-and-socket joint comprises a pin, wherein the slot and the pin are coupled to prevent circumferential rotation of the ball-and-socket joint.

12. The medical device of claim 1, wherein the link mechanism comprises a plurality of linkage bars coupled to the first jaw and the second jaw, wherein the plurality of linkage bars transfer forces from the plurality of cables to the first jaw and the second jaw.

13. The medical device of claim 1, wherein the plurality of cables each comprise a plurality of wires.

14. A system for occluding a left atrial appendage of a patient, the system comprising:
a handle;
an elongated shaft extending from the handle, the elongated shaft comprising a proximal end, a distal end, and a central lumen extending along a longitudinal axis of the elongated shaft;
an end effector at the distal end of the elongated shaft, the end effector comprising a first jaw and a second jaw configured to engage with and deliver an occlusion clip to a target tissue, wherein the end effector comprises a link mechanism configured to move the first jaw and the second jaw relative to each other;
a plurality of cables extending through the central lumen;
an articulation plate coupled to the handle;
an articulation joint coupling the articulation plate and the shaft, the articulation joint configured to allow rotational movement between the handle and the elongated shaft; and
wherein the handle comprises a lever portion configured to rotate in a first plane perpendicular to the longitudinal axis, wherein the lever portion is configured to rotate in a second plane perpendicular to the first plane, wherein rotation of the lever portion in the first plane moves the end effector in a yaw direction and rotation of the lever portion in the second plane moves the end effector in a pitch direction.

15. The system of claim 14, wherein the handle further comprises a grip portion configured for gripping by an operator with one or more fingers, wherein the articulation plate is between the grip portion and the lever portion.

16. The system of claim 14, wherein the lever portion rotates 45 degrees in a first direction and 45 degrees in a second direction in the first plane, wherein the lever portion rotates 45 degrees in a first direction and 45 degrees in a second direction in the second plane.

17. The system of claim 14, wherein the handle comprises a thumb portion extending from the lever portion, wherein the thumb portion extends at a 45-degree angle from the longitudinal axis of the elongated shaft.

18. A medical device for applying an occlusion clip, the medical device comprising:
a handle;
an elongated shaft extending from the handle, the elongated shaft comprising a proximal end, a distal end, and a central lumen;
an end effector at the distal end of the elongated shaft, the end effector comprising a first jaw and a second jaw configured to engage with and deliver the occlusion clip to a target tissue;
a plurality of cables extending through the central lumen;
an articulation plate coupled to the handle, wherein the articulation plate comprises a central opening and a plurality of peripheral openings, wherein each of the plurality of peripheral openings receives a screw;
an articulation joint coupling the articulation plate and the elongated shaft, the articulation joint configured to allow rotational movement between the handle and the elongated shaft; and
an articulation lock system within the handle, wherein the articulation lock system comprises a ring configured to engage the articulation joint and the articulation plate such that the articulation joint and the articulation plate are locked, locking the end effector.

19. The medical device of claim 18, wherein the articulation lock system further comprises a brake actuator plate, wherein the brake actuator plate is configured to release the ring from engagement with the articulation joint and the articulation plate when the occlusion clip is released.

20. The medical device of claim 19, further comprising an elastic member coupled to the brake actuator plate.
